(19) United States
(12) Patent Application Publication
(10) Pub. No.: US 2026/0294789 A1
(43) Pub. Date: Oct. 1, 2026

(54) DRY SHAMPOO RICE PAPER SHEETS

(71) Applicant: Megan Plesea, Sylvania, OH (US)

(72) Inventor: Megan Plesea, Sylvania, OH (US)

(21) Appl. No.: 19/092,059

(22) Filed: Mar. 27, 2025

(51) Int. Cl. A61K 8/9794; A61K 8/02; A61Q 5/00

(52) U.S. Cl. CPC A61K 8/9794; A61K 8/0208; A61Q 5/008; A61K 2800/87

(57) ABSTRACT

A dry shampoo product in the form of rice paper sheets coated with rice powder, designed to absorb excess oil and refresh the hair and scalp without the need for liquid shampoo. The product offers a portable, lightweight, and eco-friendly alternative to aerosol and powder-based dry shampoos. The sheets can be used by blotting and wiping directly on the scalp and hair to absorb sebum and refresh the appearance of the hair. 7 Claims, 1 Drawing Sheet.

### FIELD

The present invention relates to a disposable personal care device that absorbs sebum from the hair and scalp.

### BACKGROUND

Traditional dry shampoos are available in aerosol sprays or loose powders, both of which have drawbacks. Aerosol dry shampoos often contain propellants and chemicals that may not be environmentally friendly, while powder dry shampoos can be messy and difficult to apply evenly. There exists a need for a more convenient, mess-free, and sustainable alternative that provides effective oil absorption for hair care.

### SUMMARY

The present invention provides a dry shampoo in the form of thin rice paper sheets coated with rice powder.

The sheets are designed to be pressed or wiped against the hair and scalp, where they effectively absorb excess oil and refresh the hair without leaving visible residue.

The sheets are lightweight, disposable, and biodegradable, making them an ideal solution for on-the-go hair care.

### BRIEF DESCRIPTION OF THE DRAWINGS

FIG. 1 illustrates a perspective view of the rice paper sheets.

FIG. 2 illustrates a cross-sectional view of a single sheet showing the rice powder coating.

FIG. 3 illustrates the method of application on the scalp and hair.

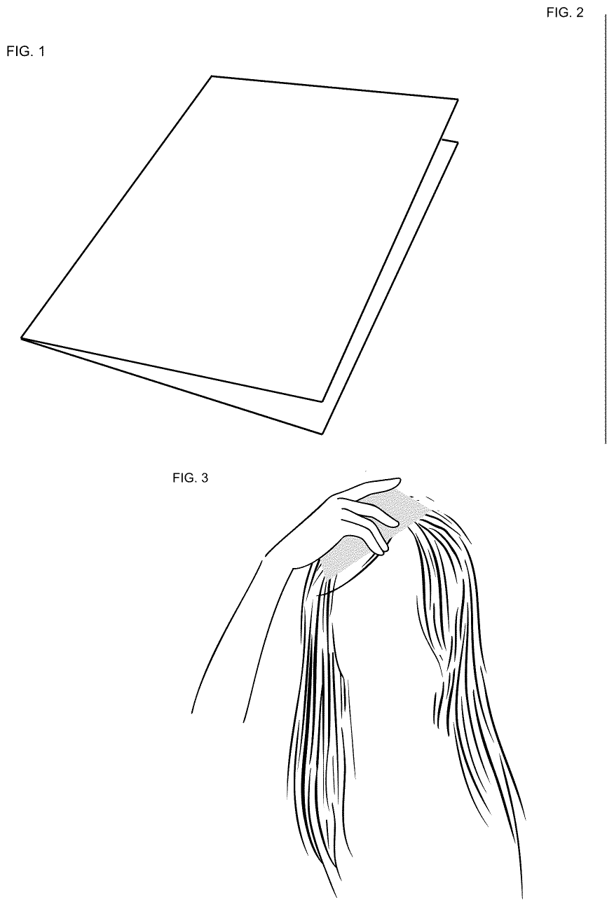

### DETAILED DESCRIPTION

The dry shampoo rice paper sheets provide a novel and improved method for absorbing excess oil from hair and scalp. With their biodegradable nature, portability, and mess-free application, they present a superior alternative to traditional dry shampoos in powder or aerosol spray forms. The invention is particularly suited for individuals seeking a sustainable and convenient hair care solution.

The dry shampoo rice paper sheets are constructed using an absorbent rice paper material that is thin, flexible, and biodegradable. The surface of each sheet is coated with a fine layer of rice powder, which enhances the oil-absorbing properties of the product. The rice powder is known for its natural ability to absorb excess sebum and leave the hair feeling fresh without any sticky or powdery residue.

To use the product, the user takes a single sheet and gently blots or wipes it on the scalp and hair, focusing on oily areas. The sheet absorbs oil on contact and can be discarded after use. The sheets are packaged in a small, portable dispenser for convenience.

The invention provides the following advantages:
1. A mess-free and convenient application process.
2. Portability, making it ideal for travel or daily use.
3. An eco-friendly and biodegradable alternative to traditional dry shampoos.
4. No need for water or aerosol propellants.

1. A dry shampoo product comprising: a) a rice paper sheet; b) a coating of rice powder on at least one surface of the rice paper sheet; wherein the coated rice paper sheet is configured to absorb excess oil from hair and scalp upon contact.

2. The dry shampoo product of claim 1, wherein the rice powder is finely milled to ensure even application and effective absorption of sebum.

3. The dry shampoo product of claim 1, wherein the rice paper sheets are individually dispensed from a portable packaging container.

4. The dry shampoo product of claim 1, wherein the sheets are biodegradable and environmentally friendly.

5. The dry shampoo product of claim 1, wherein the rice paper sheets are infused with natural fragrances or essential oils for additional hair-refreshing properties.

6. The dry shampoo product of claim 1, wherein the sheets are suitable for all hair types and leave minimal to no visible residue after use.

7. A method of reducing excess oil in hair, comprising: a) providing a rice paper sheet coated with rice powder; b) pressing or wiping the sheet against an area of the scalp or hair with excess oil; c) allowing the rice powder to absorb oil upon contact; d) discarding the used sheet after application.
